(19) United States
(12) Patent Application Publication
(10) Pub. No.: US 2026/0097146 A1
(43) Pub. Date: Apr. 9, 2026

(54) ODOR REMOVAL DEVICE, SYSTEM AND METHOD

(71) Applicant: Air+, Inc., Rye, NY (US)

(72) Inventor: Lawrence T. Sunshine, Rye Brook, NY (US)

(21) Appl. No.: 19/349,722

(22) Filed: Oct. 3, 2025

Related U.S. Application Data
(60) Provisional application No. 63/703,858, filed on Oct. 4, 2024.

(51) Int. Cl. A61L 9/22

(52) U.S. Cl. CPC A61L 9/22; A61L 2209/111; A61L 2209/14; A61L 2209/15

(57) ABSTRACT

An odor removal device, system and odor removal method(s) are provided. For example, at least one static bar is positioned between an inlet opening and an outlet opening of a housing. A fan, such as an anti-corrosive centrifugal fan, is positioned adjacent to the outlet opening in another wall of the housing. Clean air enters the housing from the inlet opening and is exposed to the charged ions from at least one static bar. The fan forces the charged ions and air into an external pipe which is aligned with the outlet opening and connected to a target. The charged ions neutralize odor within the target.

### CROSS REFERENCE TO RELATED APPLICATIONS

This application claims the benefit of U.S. Provisional Application No. 63/703,858 filed on Oct. 4, 2024, which is incorporated by reference herein in its entirety.

### FIELD

This disclosure relates to removing odors in a target environment.

### BACKGROUND

Odors are rampant in today's society. Odor is particularly problematic in the wastewater industry as there is an odor throughout the processing. Traditionally, in the wastewater industry, odor removal or control uses chemical based wet scrubbers, bio-filters or carbon adsorption systems. However, these techniques are capital intensive and require substantial maintenance. For example, the chemicals need to be regularly fed into the system. Additionally, the chemicals themselves may be hazardous, and the spent carbon may require proper disposal.

Additionally, traditional odor removal or control systems require a large footprint.

U.S. Patent Application Publication No. 2020/0062622 describes an air treatment system for a wet well. For example, FIG. 1 of the U.S. Patent Application Publication No. 2020/0062622 depicts a plurality of ionization tubes in a housing for generating ions to mix with fresh air and treat a wet well. However, like the above systems, the ionization tubes require substantial maintenance such as needing to be periodically replaced and disposed of. This may be particularly challenging depending on the location of the wet wall, as a person would need to be on-site to replace the ionization tubes. Additionally, the ionization tubes are in close proximity to each other, which leads to recombination of the charged ions prior to reaching the wet well.

### SUMMARY

Accordingly, disclosed is an odor removal system for removing odor(s) within a target. The odor removal system comprises a housing, at least one static bar and a fan, such as a centrifugal fan. The housing may be water-resistant and anti-corrosive. The housing comprises a cover and a base. The base has walls which form a compartment. The cover is configured to cover the base when closed and expose the base when opened. A wall of the base has a first opening. The first opening is an air inlet to the housing. The first opening has a screen and/or a filter installed therein. The base further comprises another wall with a second opening. The second opening provides an outlet for air to leave the housing. Each static bar is configured to produce ions. Each static bar is positioned in the airflow between the first opening and the second opening. The ion producing elements are integral to said static bar. The fan is positioned adjacent to the second opening. The fan is also attached to the another wall. The fan has a fan outlet aligned with the second opening. The fan is configured to force the ions and the air into an external pipe connected with a target to neutralize odor(s) within the target. The second opening is aligned with the external pipe providing air communication. Power is selectively supplied to each static bar and the fan, such as from an external power source.

In an aspect of the disclosure, the at least one static bar may be arranged in a first direction substantially orthogonal to the airflow within the water-resistant housing. The at least one static bar may be arranged at a non-parallel, non-orthogonal angle to the wall that has the first opening.

In an aspect of the disclosure, two or more static bars may be adjusted within the housing substantially parallel to each other. Each static bar may be arranged in a first direction substantially orthogonal to the airflow within the water-resistant housing and is offset from each other in a second direction and/or a third direction, the second direction is substantially orthogonal to the first direction and the third direction.

In an aspect of the disclosure, the odor removal system may further comprise at least one internet of things (IoT) device. Each IoT device may comprise a wireless communication interface and a sensor. Each IoT device may be configured to wirelessly communicate a sensed value to a management system. For example, the sensor may be an ion sensor configured to sense a threshold amount of ions and the wireless communication interface may transmit an indication based on a sensing to the management system. In another example, the sensor may comprise a clock configured to track a number of hours the at least one static bar is ON and the wireless communication interface may transmit the number of hours to the management system. In another example, the sensor may be a differential pressure sensor arranged and configured to detect pressure drop across the filter. In a case where the detected pressure drop is above a threshold, the wireless communication interface may transmit in indication to clean or replace the filter to the management system. In another example, the sensor may be an air quality sensor in air communication with the target and the speed of the centrifugal fan may be controlled based on a detection result from the air quality sensor.

In an aspect of the disclosure, the odor removal system may further comprise a sensor configured to detect a position of the cover and a controller configured to control the centrifugal fan to turn ON in response to the sensor detecting the position of the cover is closed. The system may further comprise a delay configured to delay the at least one static bar from turning ON until a time has elapsed from a time in which the centrifugal fan is turned ON.

In an aspect of the disclosure, the odor removal system may further comprise an air quality sensor including a hydrogen sulfide sensor positioned adjacent to the at least one static bar. When the hydrogen sulfide sensor detects hydrogen sulfide within the water-resistant housing above a threshold even after the time has elapsed, the at least one static bar may be restricted from producing ions.

In an aspect of the disclosure, the odor removal system may further comprise a temperature sensor positioned adjacent the first opening or in the external pipe. The temperature sensor is configured to sense an ambient temperature of airflow. The system may further comprise a heater. When the ambient temperature is less than or equal to a threshold, the heater may be controlled to heat the airflow until the temperature is above the threshold.

In an aspect of the disclosure, the odor removal system may further comprise a plurality of radio frequency identification devices (RFID). Each RFID device may have a sensor. In response to an interrogation by a reader, each RFID device may transmit a sensed value to the reader.

In an aspect of the disclosure, the external pipe may comprise a damper. The system may further comprise an actuator configured to control the damper to block airflow when the centrifugal fan is not turned ON.

In aspect of the disclosure, the cover nay be a door rotatably mounted to the base. The door may comprise a plurality of indicating devices including a first indicating device indicating whether the odor removal system is receiving the external power and a second indicating device indicating whether the at least one static bar is ON.

In an aspect of the disclosure, the odor removal system may further comprise a stand connectable to the wall of the base having the first opening. The stand may comprise a plurality of legs to support the water-resistant housing. The legs may not overlap the first opening.

In an aspect of the disclosure, the fan may be an axial fan.

### BRIEF DESCRIPTION OF THE DRAWINGS

FIG. 1 is a schematic illustration of an odor removal device having a single ionizing bar arranged in a first direction parallel to a wall with a first opening in accordance with aspects of the disclosure;

FIG. 2 is a schematic illustration of an odor removal device having a single ionizing bar arranged in a second direction orthogonal to a wall with a first opening in accordance with aspects of the disclosure;

FIG. 3 is a schematic illustration of an odor removal device having multiple ionizing bars arranged in a first direction parallel to a wall with a first opening and offset in both a second direction and a third direction in accordance with aspects of the disclosure;

FIG. 4A is a schematic illustration of an exterior view of a door for the odor removal device in according with aspects of the disclosure;

FIG. 4B is a schematic illustration of interior view of a door for the odor removal device in according with aspects of the disclosure;

FIG. 5 is a schematic illustration of a bottom of an odor removal device in accordance with aspects of the disclosure showing an example of a stand;

FIG. 6 is a block diagram of electronics in an internal electric box in accordance with aspects of the disclosure;

FIG. 7 illustrates a flow chart of a method of removing odor(s) in a target environment in accordance with aspects of the disclosure;

FIG. 8 illustrates a flow chart of another method of removing odor(s) in a target environment in accordance with aspects of the disclosure;

FIG. 9 illustrates a flow chart of another method of removing odor(s) in a target environment in accordance with aspects of the disclosure;

FIG. 10 illustrates a flow chart of a method of controlling the fan speed in accordance with aspects of the disclosure;

FIG. 11 is a schematic illustration of an odor removal system connected to a target environment in accordance with aspects of the disclosure mounted in a first manner;

FIG. 12 is a schematic illustration of an odor removal system connected to a target environment in accordance with other aspects of the disclosure mounted in a second manner.

### DETAILED DESCRIPTION

Aspects of the disclosure provide an odor removal device, which may be incorporated into an odor removal system and corresponding methods. The odor removal system may be used in various industrial fields such as but not limited to the wastewater industry. For example, the odor removal system may be used in headworks buildings, dewatering buildings, solid handing building, truck loading facilities, clarification systems, in processing tanks, pump and lift stations, septage receiving stations, biological system etc. The processing tanks may include wet wells, sludge storage tanks, equalization tanks and septic tanks. The odor removal system may also be used in chemical processing facilities, bathrooms, etc. The above is referred to herein as the “target environment 200”.

An odor removal system includes an odor removal device 1 and pipe(s) 205 (such as duct work). Outside (clean or fresh) air is drawn into the odor removal device 1 and interacts with ions (and ozone) produced by at least one ionizing bar 10, which may also be referred to herein as “a static bar”. The clean air with ions/ozone is blown into the pipe(s) 205 and into the target environment 200 to neutralize the odor(s). For example, the odorous and corrosive gases are oxidized on contact with the ions (and ozone) providing clean air within the target environment 200, which vents to outside the target environment.

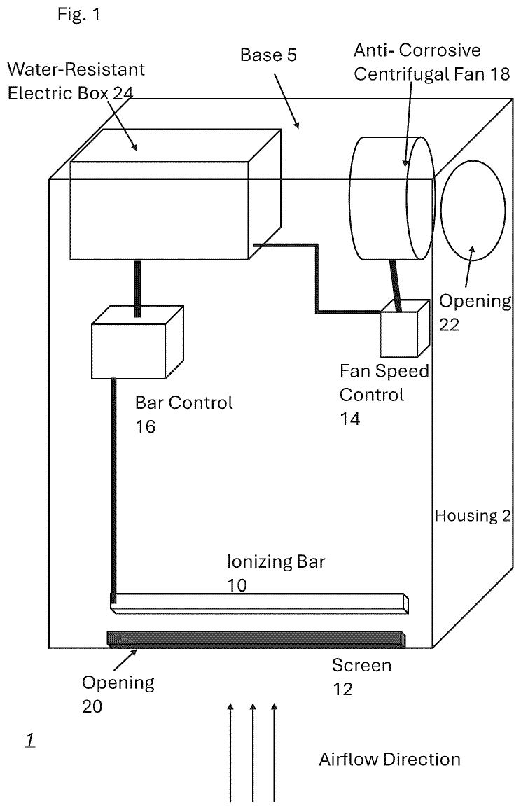

FIG. 1 illustrates a schematic diagram of an example of the odor removal device 1. The odor removal device 1 has a housing 2. The housing 2 may be formed of a material which can withstand different environmental conditions. In some aspects, the odor removal device 1 may be outside, such as proximate to a wet well or septic tanks. However, in other aspects of the disclosure, the odor removal device 1 may be within a building such as headworks buildings, dewatering buildings, and solid handling (or chemical processing plant). In other aspects, the odor removal device 1 may be installed on a rooftop such as being part of an air handler unit (AHU) and incorporated into a heating, ventilation, air conditioning system (HVAC).

Given the different environmental conditions (temperature, humidity, etc.), in an aspect of the disclosure, the housing 2 may be made of a water-resistant and anti-corrosive material. For example, the housing 2 may be a National Electrical Manufacturers Association (NEMA) type 4X electrical box. In some aspects, the housing 2 may be stainless steel. However, other NEMA types may be used such as 3R and 12 and other materials may be used. The size of the housing 2 may be target environment 200 specific and based on the volume of the airspace in the target environment 200 and application. For example, the size of the odor removal device 1 for solid handling may be different than for a wet well.

The housing 2 comprises a base 5 and a cover. In some aspects, the cover may be a door 50, which is pivotally attached to the base 5 via hinges (not shown). In other aspects, the cover may be slidably attached to the base 5 via a track or railing system. In other aspects, the cover may be attached via fasteners, screws or another attachment means. When closed, the housing 2 seals the device 1 to protect the components within the housing 2.

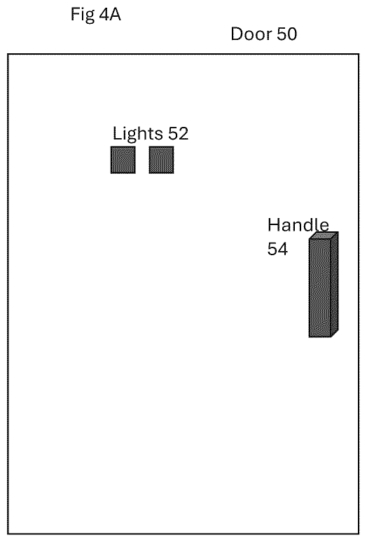

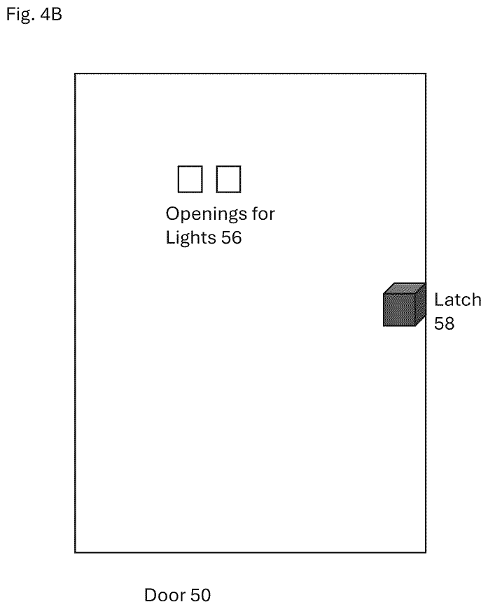

The door 50 may include a handle 54 (see FIG. 4A). The handle 54 may interact with one or more latches 58 (see FIG. 4B) on the interior side of the door 50 to lock the door (in a closed position). In other aspects, the door 50 may comprise a lock and key mechanism with a trigger or lever on the exterior surface of the door 50 instead of a handle.

The base 5 comprises sidewalls and a rear wall, which forms a compartment for the components of the odor removal device 1 (top wall, bottom wall and two walls on the sides).

One of the walls on the base has an opening 20 which is a clean/fresh air intake. The wall with the air intake opening 20 depends on the installation location and the application. FIGS. 1-3 illustrate the opening 20 on the bottom. However, in other aspects of the disclosure, the intake opening 20 may be on a different wall of the odor removal device 1. For example, when the odor removal device 1 is installed on a rooftop, the opening 20 may be on a wall on the side or the rear wall. When on a wall of the side or a rear wall has the opening 20, the opening 20 may have a protector or cover above the opening 20 to prevent inclement weather from entering the odor removal device 1. A screen 12 and/or a filter may be inserted into the opening 20. For example, a mesh screen may be inserted such as made of stainless steel. The mesh screen also acts to protect the interior of the odor removal device 1.

FIGS. 1-3 illustrate a single opening 20, however, in other aspects, there may be multiple intake openings. In some aspects, the intake openings may be on different walls of the odor removal device 1.

The filter may be an environmental filter. However, when a filter is used, the filter may need to be changed. Similarly, the mesh screen may also need to be cleaned. In some aspects, the screen 12 and/or filter may be removably attached to the odor removal device 1. For example, the screen 12 and/or filter may be removed either via the outside of the housing 2 or by opening the cover.

In some aspects, the filter may include a screen (combined screen/filter) such as a filter grill. The type of filter and screen may be application specific and size of the mesh or openings in the grill may depend on the volume of airflow needed for an odor removal system.

The odor removal device 1 comprises one or more ionizing bars 10 (static bars). A static bar is typically used in manufacturing to eliminate static charges such as on plastics, sheets, pipes, extrusions and/or cables. For example, a static bar is used in clean rooms such as in electronics manufacturing, testing and laboratories. The inventor discovered that the static bar can reduce or eliminate odor(s) in a target environment 200. Unlike ionization tubes, a static bar does not have elements that need to be periodically replaced and therefore, reduces the cost and hassle of parts replacement near the target environment 200. A static bar can both positively charge and negatively charge ions. The static bar is narrow, which enables multiple static bars to be installed in the odor removal device 1 and yet be still sufficiently spaced apart to reduce recombination of the charged ions. For example, a static bar is less than 1 inch wide.

The length of the ionizing bar 10 for the odor removal device 1 may also be application specific.

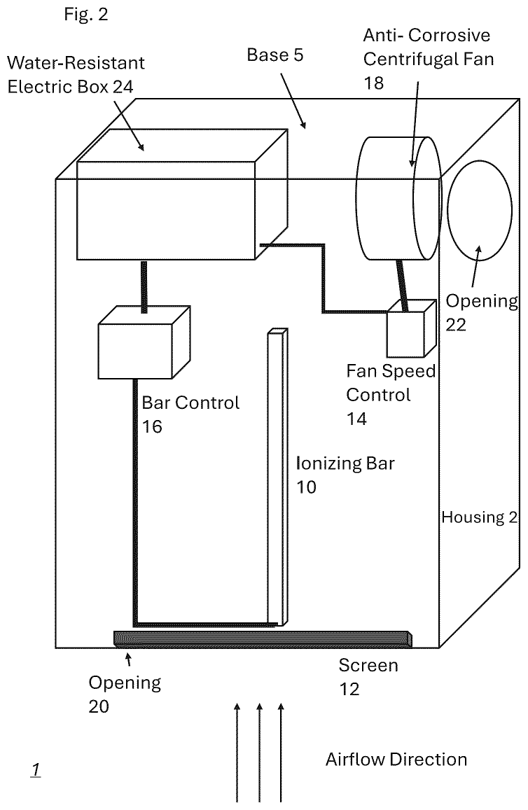

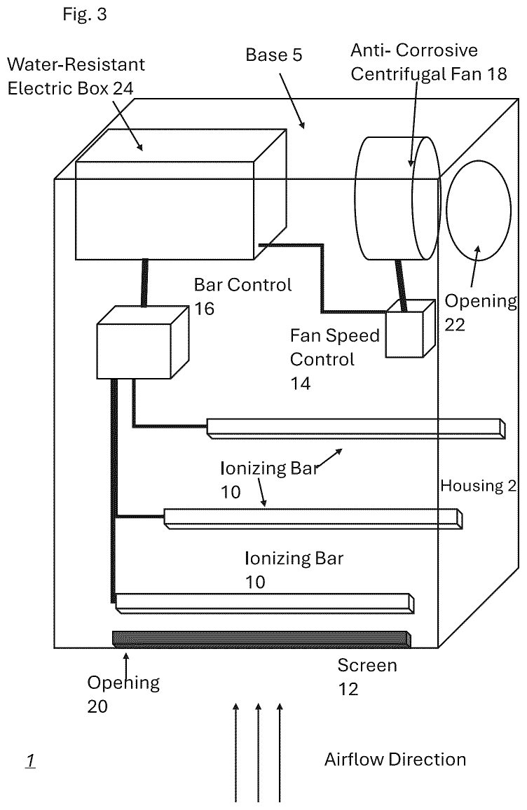

In some aspects of the disclosure, the ionizing bar 10 may be positioned in the odor removal device 1 in a first direction parallel to the wall with the opening 20 such as shown in FIGS. 1 and 3. For purposes of this description, the direction of the airflow is the main direction of the air flowing in the device 1 such as vertical in FIG. 1 In practice, the airflow is not flowing in a single direction. Additionally, depending on the relative position of the openings 20, 22, the direction of the airflow may be neither vertical nor horizontal but rather angled within the housing 2. In other aspects, the ionizing bar 10 may be positioned in the odor removal device 1 orthogonal to the wall with the opening 20 such as shown in FIG. 2 (arranged in a second direction). In other aspects, the ionization bars 10 may be angled with respect to the wall with the opening 20, e.g. neither parallel nor orthogonal to the wall with the opening 20.

In the examples depicted in FIGS. 1 and 2, one ionizing bar 10 is installed in the odor removal device 1. In other aspects, multiple ionizing bars 10 may be used. For example, FIG. 3 illustrates three ionizing bars 10 for descriptive purposes. However, the number of ionizing bars 10 is not limited to three and the number may be application specific based on the size of the odor removal device 1, the size of the opening 20 and the size of the target environment 200 and the type of the target environment 200.

In an aspect of the disclosure, when multiple ionizing bars 10 are used, adjacent ionizing bars 10 may be staggered in two directions. For example, each ionizing bar may be arranging in a first direction parallel to the wall with the opening 20. The ionizing bars 10 may also be parallel to each other. Additionally, each ionizing bar 10 may also be offset in a second direction and a third direction. The offset reduces a likelihood of recombination of the charged ions. For example, the ionizing bars 10 may be arranged at different heights (second direction) in the odor removal device 1. Additionally, the ionizing bars 10 may be arranged at different positions in the front-back direction (third direction). The spacing in the third direction may be defined by the size of the opening 20 in the third direction. In an aspect of the disclosure, the ionizing bar(s) 10 are at least partially aligned with the opening 20 (as view from the second direction) such that the entire ionizing bar 10 is exposed to the airflow. For example, if the opening is X inches front to back (third direction), the ionizing bar(s) may be positioned within the X inches in the third direction. In some aspects of the disclosure, adjacent ionizing bars 10 may be equally spaced apart.

In other aspects of the disclosure, the multiple ionizing bars 10 may be arranged in the second direction orthogonal to the wall with the opening 20 (not shown). Like above, the ionizing bars 10 may be still parallel to each other and equally spaced apart in two directions. For example, each ionizing bar 10 may also be offset in the first direction and the third direction.

This multi-dimensional separation arrangement of the multiple ionizing bars 10 reduces recombination of the ions within the odor removal device 1 and pipe 205 (duct worked) which enables a majority of the ions to reach the target environment 200 to neutralize the odor(s).

In other aspects, the multiple ionizing bars 10 may be offset only in one direction. For example, each ionizing bar 10 may be arranged in the first direction. Like above, each ionizing bar 10 may also be parallel to each other. The ionizing bars 10 may be offset in the third direction (front-back direction). Each ionizing bar 10 may be also arranged in the second direction and parallel to each other. The ionizing bars 10 may be offset in either the first direction or the third direction (front-back direction) in this configuration.

In some aspects, the minimum spacing being adjacent ionizing bars 10 may be based on the application, the size of the odor removal device 1, the size of the opening 20 and the size of the target environment 200 and the type of the target environment 200, and the number of ionizing bars 10 and set to reduce recombination of the charged ions within the odor removal device 1 or pipe 205.

The ionizing bars 10 may be arranged in different patterns such as a zigzag which one end of adjacent ionizing bars 10 is closed than the other end of adjacent ionizing bars.

The terms “parallel” and “orthogonal” include substantially parallel and substantially orthogonal. In other aspects, the ionizing bars 10 may be positioned at different angles with respect to the airflow within the odor removal device 1.

The ionizing bar(s) 10 may be mounted to the base 5 using a series of mounting brackets. For example, a portion of a mounting bracket may be attached to a wall of the odor removal device 1 and another portion extends into the compartment to hold the ionizing bar(s) 10. The brackets may be different lengths to enable the offset as described above. In some aspects, the mounting brackets may be adjustable to change the distance from a wall and/or tilt. Depending on the arrangement, the brackets may be attached to different walls. The brackets may be positioned to minimize the blocking of the airflow.

The ionizing bar(s) 10 are electrically connected to a controller (bar control 16). In an aspect of the disclosure, the same controller may be used for multiple ionizing bars. In this aspect, the ionizing bars 10 may produce about the same output. The bar control 16 may be fixed to the rear wall of the odor removal device 1. The bar control 16 is connected to one or more components in the internal electric box 24.

The odor removal device 1 also has an outlet opening 22. The outlet opening 22 may be on another wall. For example, in some aspects, the outlet opening 22 may be higher than the intake opening 20. As depicted in FIGS. 1-3, the opening 22 is on a wall on the side of the odor removal device 1. Also as depicted, the wall with opening 22 is orthogonal to the wall with the opening 20. However, the wall with opening 22 need not be orthogonal to the wall with the opening 20. For example, in some aspects, the openings 20, 22 may be aligned as viewed from the first direction or the third direction. Aligning the openings 20, 22 may shorten the distance the ions need to travel within the odor removal device 1. The position of the opening 22 may be based on the location of the target environment 200 (relative to the odor removal device 1) and available placement position for the odor removal device 1.

The odor removal device 1 has a fan mounted on the same wall as the outlet opening 22. In some aspects, the fan is a centrifugal fan 18 with a casing which is anti-corrosive. Once again, the fan may be exposed to corrosive gases, and the casing needs to be resistant to corrosion. The benefit of using a centrifugal fan 18 is that the incoming airflow may be a different direction than the outgoing airflow. As shown in FIGS. 1-3, the incoming airflow may be generally vertical, and the outgoing airflow may be generally horizontal (in the depicted orientation). However, other types of fans may be used such as where the openings 20, 22 are aligned. In a case where the openings 20, 22 are aligned, an axial fan may be used.

The outlet (air outlet) of the centrifugal fan 18 is mounted to the wall with the opening 22 and at least partially aligned with the opening 22. For example, the outlet of the centrifugal fan may have a rectangular shape and the opening 22 may be circular. In an aspect of the disclosure, the outlet of the centrifugal fan and the opening may be substantially the same size. The inlet of the centrifugal fan may be arranged to face the incoming airflow.

In some aspects, the odor removal device 1 is connected to the target environment 200 via a pipe 205 (duct work). The pipe 205 may be connected to the odor removal device 1 via a collar 210. The collar 210 is larger than the opening 22. The pipe 205 is mounted to the base 5 via the collar 210 such that the opening of the collar 210 aligns with the opening 22 and the outlet of the centrifugal fan 18. In other words, the wall with the opening 22 is between the collar 210 and the outlet of centrifugal the fan 18. The collar 210 is attached to the odor removal device 1 and maintains an airtight seal which enables a control of the airflow into the target environment 200. In other aspects, when a different fan type is used, such as an axial fan, the rim of the fan may be mounted to the wall with the opening 22.

The odor removal device 1 has a fan speed control 14. The fan speed control 14 may be mounted to the rear wall of the odor removal device 1 and positioned adjacent to the centrifugal fan 18. The fan speed control 14 is also connected to one or more components of the internal electric box 24. The fan speed control 14 electrically connects the centrifugal fan 18 to an external power source and regulates the power to the centrifugal fan 18. The fan speed control 14 may also comprise an ON/OFF switch. In other aspects, a separate ON/OFF switch may be used. The fan speed control 14 is connected in series with the centrifugal fan 18.

In an aspect of the disclosure, the fan speed control 14 may be manually set to a speed. In other aspects, as described later, the speed may be automatically adjusted based on a detection of odor(s).

The odor removal device 1 also has an electric box 24 configured to hold one or more electric components of the device 1. The electric box 24 is water-resistant and anti-corrosive. In some aspects, the electric box 24 may be stainless steel. For example, the electric box 24 may be a NEMA type 3R. The electric box 24 may also be opened such as by removing its cover to access the one or more electric components.

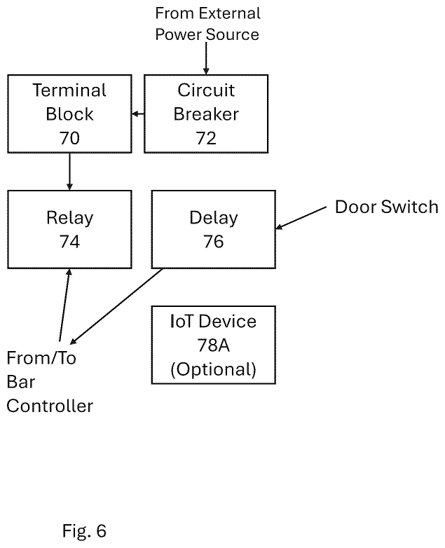

FIG. 6 illustrates a block diagram illustrating examples of the one or more electric components within the electric box 24. These components may include a terminal block 70, a circuit breaker 72, a relay 74 and a delay 76.

The odor removal device 1 may be powered by an external power source. The external power source may supply AC power such as 120 VAC. In some aspects, the external power source may be local grid power. Depending on the location, the local grid power may be different than 120 VAC. In some aspects, a power converter may be included in the odor removal device 1. The base 5 of the housing 2 of the odor removal device 1 and the electric box 24 may have an opening for wires to be inserted into which are connected to the external power source. These openings may be weather shielded. Additionally, the openings may be aligned to minimize the length of the wire (cable) needed. The wires from the external power source may be connected to the circuit breaker 72. The circuit breaker 72 may be connected to a terminal block 70. One or more of the components described above may be directly or indirectly connected to the terminal block 70.

In an aspect of the disclosure, the odor removal device 1 may comprise a door switch. The door switch may act as a main power switch to prevent an operator's exposure to the high voltage components of the ionizing bar(s) 10 and access to a rotating fan blade. For example, when the door 50 is opened, the door switch does not allow power to be supplied to the bar control 16 (and the ionizing bar(s) 10) and the fan speed control 14 (and the fan). Once the door 50 is closed, the door switch allows power to be supplied to the bar control 16 and fan speed control 14 subject to other determinations.

In some aspects, the door switch may be a plunger-type. The plunger may be installed on one of the walls of the base in a position to interact with the door 50. The plunger makes contact with the door 50 when the door 50 is closed and does not make contact with the door 50 when the door 50 is opened. In other aspects, other types of switches may be used as the door switch including pressure switches, optical switches and magnetic switches

The door switch may be connected in series with the circuit breaker 72.

In an aspect of the disclosure, the delay 76 is connected in series with the door switch. The delay 76 is configured to restrict the ionizing bar(s) 10 from being turned ON until the centrifugal fan 18 is running for a period of time. The delay 76 functions to (1) allow any pollutant such as hydrogen sulfide to be evacuated from the odor removal device 1 by the centrifugal fan; and (2) limit a build-up of ions and ozone within the odor removal device 1. The fan 18 draws air from within the device 1 and blows into the pipe 205. The delay reduces a likelihood of a combustible event. For example, certain pollutants, e.g., hydrogen sulfide, are flammable, depending on the concentration. The ionizing bar(s) 10 should not turn ON at the same time or before the centrifugal fan 18. Since the centrifugal fan 18 and fan speed control 14 are in series with the door switch, the fan will turn ON when the door 50 is closed, which allows the fan 18 to run prior to the ionizing bar(s) 10 producing ions (because of the delay). Even if the door 50 is closed, the ionizing bar(s) 10 does not produce ions until the delay expires. The amount of the delay (time offset) may be application specific. For example, in some aspects, the delay may be 5 seconds. In other aspects, the delay may be 10 seconds. In yet other aspects, the delay may be longer. The delay may be determined based on an expected exposure volume of the pollutants, e.g., hydrogen sulfide.

In other aspects of the disclosure, the delay 76 may be keyed off of the fan 18 rather than the door switch. For example, the fan 18 may not turn ON even if the door is closed such as if the fan 18 is not working. In some aspects, there may be a sensor detecting the speed of the fan and if the speed reaches a threshold, the delay starts.

In some aspects of the disclosure, the door 50 of the odor removal device 1 may include one or more lights 52 (as shown in FIG. 4A). The lights 52 provide an indication of the operation of the odor removal device 1. The lights 52 may be connected to the external power source via a relay 74. FIG. 4A illustrates two lights 52 and FIG. 4B illustrates two openings for the lights (openings 56) for illustrative purposes. One light may be used to indicate that the odor removal device 1 is receiving power, e.g., POWER ON. Another light may be used to indicate the status of the fan (e.g., centrifugal fan 18) (ON or OFF). In other aspects, a light may be used to indicate the status of the ionizing bar(s) 10. In some aspects, multiple lights 52 may be used for the ionizing bars 10, one respectively for each of the ionizing bars, when multiple ionizing bars are used. Each light 52 is viewable from the exterior of the cover such that a person does not need to open the cover to understand the status. When multiple lights 52 are installed in the cover, the cover may have text to indicate the function of each light. In some aspects, the color of the lights 52 may be different such that the function may be easily differentiated by the color. In an aspect of the disclosure, each light 52 may be a light emitting diode (LED).

In some aspects of the disclosure, the odor removal device 1 may also comprise an air quality sensor (such as incorporated in an air quality switch). In this aspect of the disclosure, the air quality switch may be connected in series with the delay 76. Even after the delay, the amount of pollutant may still be above a concentration which is safe to start producing charged ions by the ionizing bar(s) 10. Multiple air quality sensors (and its associated air quality switch) may be used for different pollutants. At least one of the air quality sensors/switches may be for hydrogen sulfide. Additionally, in some aspects, the turn ON thresholds may be different for different pollutants (where the ionizing bars may be turned ON when the concentration for the respective pollutant with the odor removal device 1 is less than its respective ON threshold.

In an aspect of the disclosure, the odor removal device 1 may also comprise one or more IoT devices 78A-78N, where N is the number of IoT devices. Each IoT device is configured to communicate with a management system such as a Supervisory Control and Data Acquisition (SCADA). The SCADA may monitor the operation of the odor removal device 1 via the one or more IoT devices 78A-78N. Each IoT device may comprise a wireless communication interface and a sensing element.

For example, an IoT device (e.g., 78A) may be incorporated into the electric box 24. In some aspects, this IoT device 78A may include a current or voltage sensor for monitoring power in the odor removal device 1 (e.g., is the device ON). Similarly, other IoT devices may be connected to the ionizing bar(s) 10 or the centrifugal fan 18 to monitor the status of the same. Each IoT device reports the status to the SCADA. Other IoT devices may include different sensors including, but not limited to, an ion detector, pressure sensor, temperature sensor, speed sensor (as described herein) to respectively report sensed values to the SCADA.

For example, an ion detector (associated with an IoT device) may be positioned downstream of the ionizing bar(s) 10 and set at a threshold. If the ions produced by the ionizing bar(s) 10 drop below the threshold, the IoT device may transmit the status to the SCADA. This drop in ions produced may indicate that the ionizing bar(s) needs to be cleaned. Alternatively, the IoT device may report the status when the detected ions are above the threshold and stop when below the threshold. In other aspects, the IoT device may continuously report an amount of ions detected.

An operator at the SCADA using the detections may dispatch a technician for cleaning. In some aspects, the ion detector may be a duct ion sensor installed through a wall of the base 5. In other aspects, a duct ion sensor may be installed in the pipe 205 connecting the odor removal device 1 and the target environment 200.

Additionally, a pressure sensor (associated with an IoT device) may be positioned adjacent to the screen 12 and/or filter and monitor the pressure change. A change in pressure (or lack thereof) may indicate that the screen 12 and/or filter may need to be cleaned or replaced. In some aspects, the pressure sensor may be a differential pressure sensor and there may be a pressure difference threshold which may trigger the indication. The IoT device may transmit the pressure status to the SCADA such as an indication that the pressure. An operator at the SCADA may dispatch a technician for cleaning or replacement.

In other aspects, an IoT device may have a clock/timer that tracks the ON time for the ionizing bar(s) 10 and the centrifugal fan 18 and the IoT device may transmit the time to the SCADA. The ON time may also indicate that cleaning or maintenance may be needed for the ionizing bar(s) 10 and the centrifugal fan 18. An operator at the SCADA may dispatch a technician for cleaning or replacement.

An IoT device with a speed sensor may be used to confirm that the centrifugal fan 18 is providing the controlled speed by the fan speed control 14. Any difference may indicate the fan 18 and/or fan speed control 14 is malfunctioning.

The IoT devices, as described above, enable a timely scheduling of cleaning and maintenance and eliminate wasted on-site trips and also ensures proper functioning of the components.

In an aspect of the disclosure, the system may include a temperature sensor. The temperature sensor may be positioned within the odor removal device 1 or in the pipe 205. In some aspects, the temperature sensor may be positioned adjacent to the screen 12 and/or filter.

The temperature sensor is configured to detect the ambient temperature of the airflow. In this aspect of the disclosure, the system may also include a heater. In some aspects, the heater may be a resistive heating element. The heater may also be positioned within the odor removal device 1 or in the pipe 205. The temperature sensor/heater may be incorporated into a system which is used outside. The heater may be turned ON by a sensor control switch when the temperature is below or equal to a threshold. The sensor control switch may be a line voltage thermostat. The threshold may be set to prevent the odor removal device 1 from blowing cold air into the target environment 200, such as when the temperature is below freezing and could damage the target environment 200. In some aspects, the system may include a timer to control the time in which the heater is ON. For example, the timer may be set to a predetermined time. In other aspects, the time may be varied based on the detected temperature. For example, the threshold may be 32 degrees. If the detected temperature is 20 degrees, the time may be longer than if the detected temperature is 25 degrees.

In an aspect of the disclosure, the fan speed control 14 may communicate with an air quality sensor in the target environment 200. The air quality sensor may also be incorporated into an IoT device 78B (or another wireless device). However, depending on the target environment, in other aspects of the disclosure, the air quality sensor may be wired to the odor removal device 1 instead of an IoT device. In this aspect, the air quality sensor may report the sensed values to the fan speed control 14 and the fan speed control 14 may communicate with the SCADA via an-on board IoT device. The air quality sensor may provide control feedback for the fan speed control 14 to automatically adjust the fan speed. For example, while the odor removal device 1 is ON and the centrifugal fan 18 is blowing air at a first speed, the air quality sensor reports the sensed values. If there is no change or not a sufficient change, the fan speed control 14 may change the speed of the fan. A faster fan speed may reduce the recombination of the charged ions within the pipe 205 because the airflow is moving faster. However, the faster airflow may cause the charged ions to be vented from the target environment 200 prior to neutralizing the odor(s). A slower fan speed may allow the charged ions to remain in the target environment 200 longer allowing time to neutralize the odor(s). Therefore, in some aspects of the disclosure, the change in air speed may be application specific, based on the diameter of the pipe 205, the length of the pipe 205, and the size of the target environment 200.

In accordance with this aspect of the disclosure, the fan speed control 14 may include a processor which may increase or decrease the fan speed in accordance with the feedback from the air quality sensor in the target environment 200. In some aspects, the amount of the change of the fan speed may be based on a stored look up table in a memory in the fan speed control 14.

The air quality sensor may be application specific or target environment specific and different volatile organics sensors may be used. In some aspects, the air quality sensor is a hydrogen sulfide sensor.

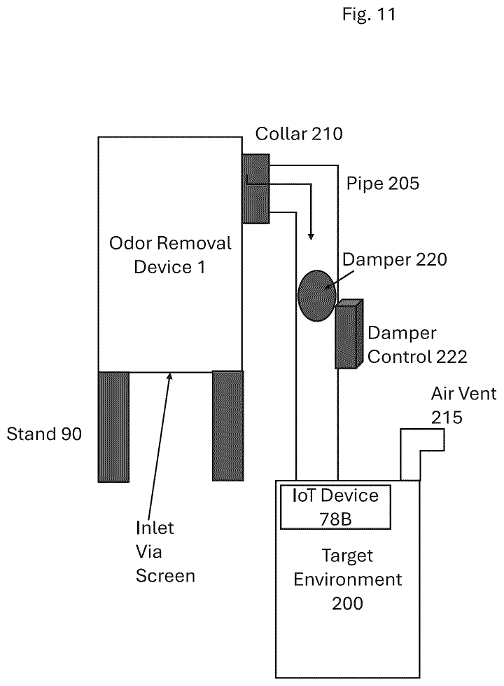

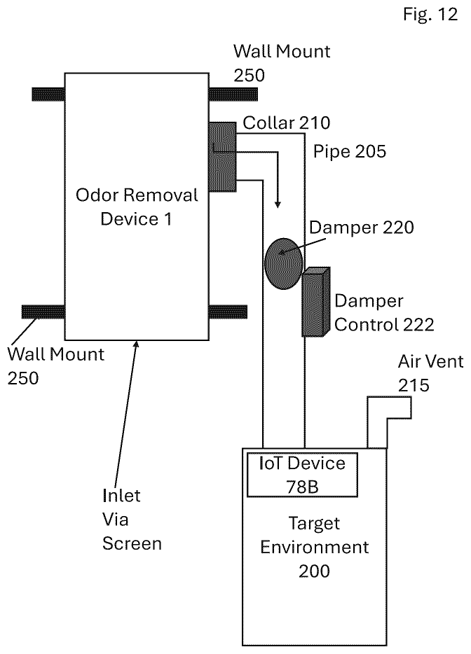

In some aspects of the disclosure, the system may comprise a damper 220 installed in the pipe 205. The damper 220 may be used in certain applications and for certain target environments 200. The damper 220 may be positioned in the pipe 205 between the odor removal device 1 and the target environment 200. The damper 220 is schematically shown in FIGS. 11 and 12 in the pipe 205. The damper 220 may comprise a rotatable blade(s) with a gasket so that the damper is airtight.

The damper 220 may be positioned downstream of the collar 210. In some aspects, one end of the damper 220 may be connected to the collar 210. When closed, the blade(s) with the gasket has the same diameter as the interior diameter of the pipe 205.

In some aspects, the rotation of the blade(s) is/are controlled by a damper control 222. The damper control 222 may include an actuator. The damper controller 222 may be mounted to the pipe 205 such as shown in FIGS. 11 and 12. The pipe 205 has an opening for the actuator to enter into the internal space of the pipe 205 and contact the blade(s). For example, the actuator may be mechanical connected to a shaft of the blade(s) to open or close the blade(s). When the blade(s) is closed, airflow in both directions is blocked, whereas when the blade(s) is opened, air may flow (in either direction).

In some aspects, a control for the actuator is electrically connected to the odor removal device 1. The damper control 222 may comprise a housing. The housing may be water-resistant and anti-corrosive. For example, the housing may be made from stainless steel and be a NEMA type 4X box.

The housing may have an opening for a connection cable to be inserted and connected to the housing 2 of the odor removal device 1. The housing 2 of the odor removal device 1 may also have a corresponding opening for the cable. In some aspects, each opening may be weather shielded if needed.

Within the housing 2 of the odor removal device 1, the cable may be electrically connected to the fan speed control 14 and specifically to an ON/OFF switch thereof (or a separate switch). Since the ON/OFF switch (or the fan speed control 14) is electrically connected to the external power source, this connection (1) supplies power to the damper control 222 and (2) forms a detection that the fan 18 is ON, which doubles as power and as a control signal. When the centrifugal fan 18 is ON, the control causes the actuator to open the blade(s) and when the centrifugal fan is OFF, the control causes the actuator to close the blade(s).

In some aspects of the disclosure, additionally or alternatively, a speed sensor or a pressure sensor may be positioned upstream of the damper 220 to detect the motion of the fan (or pressure change caused by the air moving). The sensor may be electrically connected to the damper control 222. In this aspect of the disclosure, the control causes the actuator to open/close based on the detection results from the sensor.

In some aspects of the disclosure, the damper control 222 may have its own external power source. For example, the damper control 222 may be directly connected to the grid in a similar manner to the odor removal device 1. The sensor may obtain power from the damper control 222. In some aspects, the damper control 222 may have a battery back up (or even a battery as a primary power source). In some aspects, the damper control 222 may include a solar panel on the housing and the battery may be rechargeable by the solar panel such as via a converter.

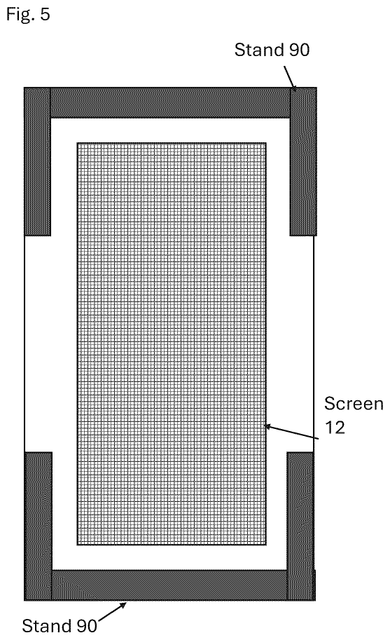

Depending on the application and the target environment 200, the odor removal device 1 may be mounted using different types of mounting. For example, where the odor removal device 1 is attached to a rooftop of a building or attached to the ground, the odor removal device 1 may be mounted via a stand 90 such as shown in FIG. 11. The stand 90 may comprise a plurality of legs attached to the bottom corners of the odor removal device 1. FIG. 5 illustrates a schematic bottom view of the odor removal device 1. As shown, the legs do not block the screen 12, providing a clear path for ambient air to enter the odor removal device 1. FIG. 5 shows two legs, however, there may be four legs. The legs may be bolted to the rooftop of a building or the ground. In some aspects, depending on the terrain, the stand 90 may also include a cement base or block.

In other aspects, the odor removal device 1 may be wall-mounted. In an aspect of the disclosure, the odor removal device 1 may have an integral field mount bracket(s) preinstalled on the housing 2. In other aspects, the odor removal device 1 may be mounted using tracks such as unistruts to a wall adjacent to the target environment 200 such as shown in FIG. 12. As illustrated in FIG. 12, there are two wall mounts 250: one on the top and one on the bottom (both attached to the rear wall).

FIGS. 11 and 12 illustrate alternative mountings for an odor removal system in accordance with aspects of the disclosure. As shown, the target environment 200 may be a tank such as a wet well. Clean air enters the odor removal device 1 via the screen 12. The centrifugal fan 18 blows ionized air (ions provided by the ionizing bar(s) 10) to the pipe 205 into the target environment 200. The target environment 200 has an air vent 215 which is a path for the odor-removed air to flow (e.g., vent).

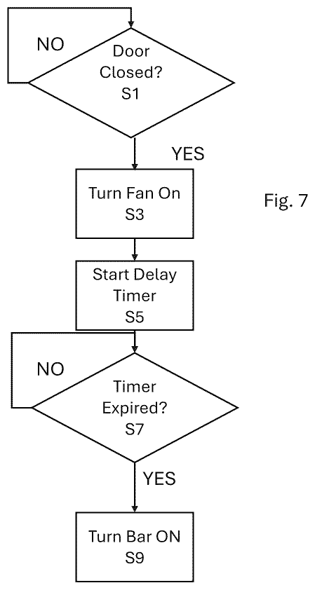

FIG. 7 illustrates a flow chart of a method of removing odor(s) from a target environment 200 in accordance with aspects of the disclosure using an odor removal system having an odor removal device 1 in accordance with aspects of the disclosure.

At S1, it is determined whether the cover such as a door 50 is closed. When the cover is opened, neither the centrifugal fan 18 nor the ionizing bar(s) 10 are ON. The determination in S1 may be made using a door switch. When it is determined that the cover is closed (“YES” at S1), the centrifugal fan 18 is allowed to be turned ON at S3. For example, since the fan speed control 14 (switch) may be connected in series with the door switch, power is supplied to the fan speed control 14 and the centrifugal fan 18 is able to rotate. In some aspects of the disclosure, where there is a damper 220 in the pipe 205, the damper control 222 also opens the damper 220 in response to the cover being closed and the centrifugal fan 18 turning ON. Once the centrifugal fan 18 is turned ON, the delay is started at S5 (delay 76). For example, the delay starts the time. In some aspects, the delay is set to 5 seconds. At S7, the delay 76 determines whether the set time has expired. When the set time has expired (e.g., 5 seconds), the ionizing bar(s) 10 are allowed to be turned ON at S9. For example, power may be supplied to the bar control 16, which controls the ionizing bar(s) 10 to turn ON. When the set time has not expired, the power is not supplied to the bar control 16 (“NO” at S7). In other aspects, the delay may be set based on the application and the type of target environment 200.

The centrifugal fan 18 and the ionizing bar(s) 10 may be maintained in an ON state until the cover is opened. When the cover is opened, the door switch isolates power from the bar control 16 and the fan speed control 14 (including the ON/OFF switch) and the ionizing bar(s) 10 and centrifugal fan 18 are turned OFF. In some aspects, there may be a reversed delay such that the ionizing bar(s) 10 is turned OFF before the centrifugal fan 18.

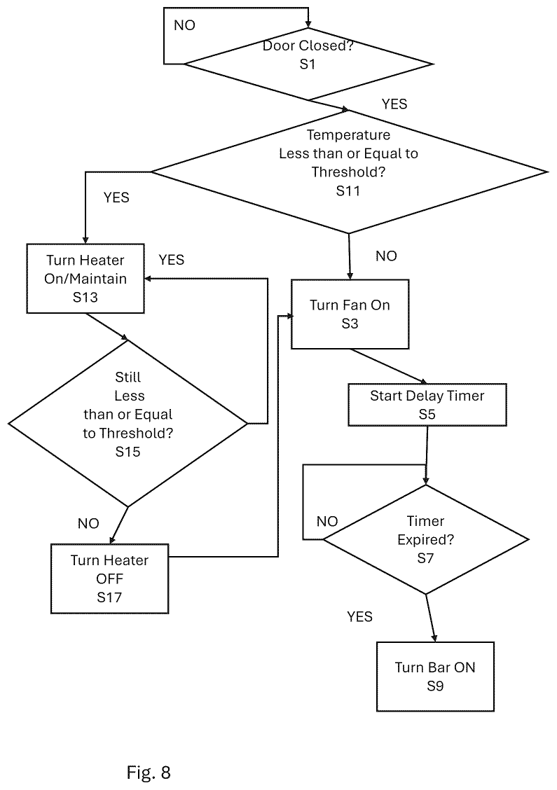

FIG. 8 illustrates a flow chart of another method of removing odor(s) from a target environment 200 in accordance with aspects of the disclosure using an odor removal system having an odor removal device 1 in accordance with aspects of the disclosure. This method may be used when the target environment 200 is outside and exposed to weather conditions.

At S1, it is determined whether the cover such as a door 50 is closed. When the cover is opened, neither the centrifugal fan 18 nor the ionizing bar(s) 10 are ON. The determination in S1 may be made using the door switch. When it is determined that the cover is closed (“YES” at S1), the centrifugal fan 18 is enabled to be turned ON. However, in this method, before the centrifugal fan 18 is turned ON, the ambient temperature is detected using one or more temperature sensors. At S11, a determination is made whether the ambient temperature is less than or equal to a threshold. The determination may be made using a line voltage thermostat. In response to the temperature being less than or equal to the threshold (“YES”) at S11, a heater is controlled to turn ON at S13. The temperature is continuously monitored at S15 such as by the line voltage thermostat. The heater is maintained to be ON until the temperature exceeds the threshold (“YES” at S15). When the temperature exceeds the threshold (“NO” at S15), the heater is turned OFF at S17 by the line voltage thermostat. Afterwards, the centrifugal fan 18 is turned ON at S3. The remaining flow of the method is the same as in FIG. 7. This method avoids blowing cool air into the target environment 200. For example, when the target environment 200 is a wet wall, blowing cool air may freeze components within the well.

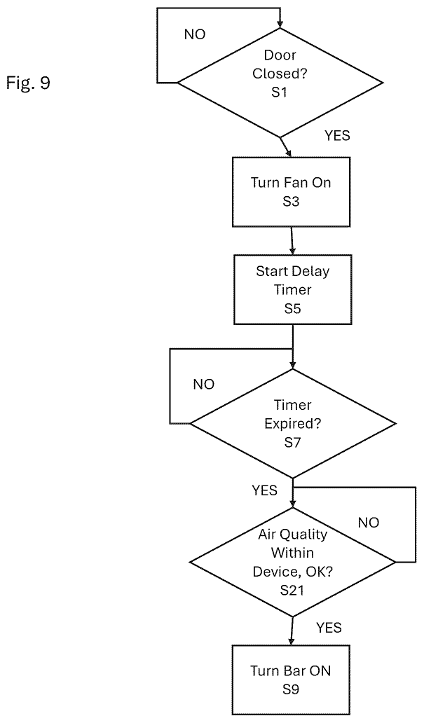

FIG. 9 illustrates a flow chart of another method of removing odor(s) from a target environment 200 in accordance with aspects of the disclosure using an odor removal system having an odor removal device 1 in accordance with aspects of the disclosure. The method illustrated in FIG. 9 is similar to the method illustrated in FIG. 7 except that prior to turning the ionizing bar(s) ON, it is determined whether the air quality within the odor removal device 1 is within a target range for each monitored pollutant. This check forms another safety check. The ionizing bar(s) 10 are only allowed to turn ON (S9) when the monitored pollutants are within an acceptable range, e.g., below a respective ON threshold (“YES”) at S21. One or more air quality switches enable/disable the ionizing bar(s) 10 from turning ON. The ionizing bar(s) 10 are maintained OFF with the centrifugal fan 18 running as long as at least one monitored pollutant is above the respective ON threshold (“NO”) at S21.

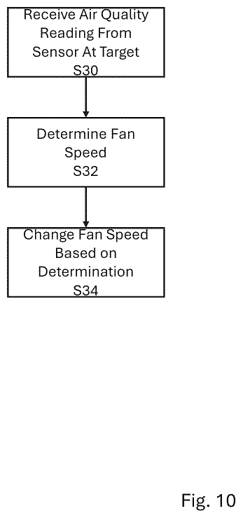

FIG. 10 illustrates a flow chart for controlling the fan speed while the centrifugal fan 18 is running and the ionizing bar(s) 10 are producing ions in accordance with aspects of the disclosure. In accordance with aspects of the disclosure, the fan speed control 14 may automatically adjust the fan speed based on feedback from air quality sensor(s) such as incorporated in an IoT device 78B within the target environment 200. At S30, the fan speed control 14 receives the sensor reading(s) from air quality sensor(s). In some aspects, the fan speed control 14 compares the sensor reading(s) from a prior sensor reading(s), respectively. In this aspect, the fan speed control 14 stores sensor reading(s) in a memory. When a sensor reading indicates that the odor(s) is/are not improving, e.g., the specific pollutant is not being neutralized, the fan speed control 14 may determine that the fan speed needs to be adjusted. At S32, the fan speed control 14 determines the adjustment amount. In some aspects, the memory has a LUT of the adjustment amounts based on the received sensor reading(s). The fan speed control 14 looks up the adjustment amount and changes the speed based on the obtained amount at S34.

In some aspects of the disclosure, the fan speed control 14 comprises a processor such as a programmable logic controller to execute the functionality shown in FIG. 10.

In some aspects of the disclosure, the odor removal device 1 may additionally have or alternatively (to the IoT device(s)), radio frequency identification devices (RFID) respectively associated with each sensor. When an operator or a technician is on-site, a reader may be used to interrogate each RFID to read out the sensed values to obtain the operating state of the odor removal device 1.

In an aspect of the disclosure, the bar control 16 may have a fixed setting for the ionizing bar(s), e.g., set at maximum output. However, in other aspects of the disclosure, each ionizing bar 10 may be controlled to adjust the output such as based on an air quality sensing at the target environment 200. The bar control 16 may also comprise a PLC or other processing hardware.

The size, both in diameter and length of the pipe 205 may also impact the availability of charged ions to neutralize odors within the target environment 200. The size of the pipe 205 may be application specific and based on the size of the target environment 200. For example, a narrow pipe (in interior diameter) and a long pipe 205 may enable the charged ions to recombine prior to reaching the target environment. However, a wider pipe (in interior diameter) allows for the charged ions to spread out over the diameter and achieves less recombination. Similarly, a short pipe 205 may also lead to less recombining.

In some aspects of the disclosure, the odor removal device 1 may be powered using solar power. In this aspect of the disclosure, solar panel(s) may be installed on the top of the housing 2 and positioned to face the sun. The direction may vary depending on the location of the device 1. The odor removal device 1 may also include an internal power device configured to receive the solar power and convert as needed to a line voltage for the odor removal device 1. For example, the solar panel(s) may generate 12 VDC and a power converter may convert the input DC to AC, such as to 120 VAC (or 240 VAC). Prior to conversion, the solar power may be stored in an energy storage device such as a rechargeable battery.

In some aspects of the disclosure, the odor removal device 1 may be remotely controlled. For example, the SCADA based on sensed data it receives from the IoT devices may control the fan 18 (ON/OFF), speed of the fan or the ionizing bar(s) 10 such as ions production count.

In other aspects, a remote controller (remote control device) may be used to control the odor removal device 1, e.g. via infrared signals. The remote control device may have an ON/OFF button for the odor removal device 1, a speed control and ion control.

In other aspects, the odor removal device 1 may be controlled based on a time of day.

The term “about” used herein indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein. For example, the term about when used for a measurement in mm, may include +/0.1, 0.2, 0.3, etc., where the difference between the stated number may be larger when the state number is larger. For example, about 1.5 may include 1.2-1.8, where about 20, may include 19.0-21.0

The term “substantially”, or “substantial” as used herein, is equally applicable when used in a negative connotation to refer to the complete or near complete lack of an action, characteristic, property, state, structure, item, or result.

When referring to an orientation that is “substantially” in that orientation, it refers to either completely in that orientation, or nearly in that orientation that the effect would be the same as if it were completely in that orientation. “Substantially” when referring to a shape or size may account for manufacturing where a perfect shapes, such as circular or sizes may be difficult to manufacture. As used herein terms such as “a”, “an” and “the” are not intended to refer to only a singular entity, but include the general class of which a specific example may be used for illustration. As used herein, terms defined in the singular are intended to include those terms defined in the plural and vice versa.

References in the disclosure to “one aspect”, “certain aspects”, “some aspects” or “an aspect”, indicate that the aspect(s) described may include a particular feature or characteristic, but all aspects of the disclosure may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to affect such feature, structure, or characteristic in connection with other aspects whether or not explicitly described. For purposes of the description hereinafter, the terms “upper”, “lower”, “right”, “left”, “vertical”, “horizontal”, “top”, “bottom”, and derivatives thereof shall relate to a device relative to a ground and/or as it is oriented in the figures.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “processor” or the term “controller” may be replaced with the term “circuit” such as an ASIC or PLC. The term “processor” may refer to, be part of, or include processor hardware (shared, dedicated, or group) that executes code and memory hardware (shared, dedicated, or group) that stores code executed by the processor.

The corresponding structures, materials, acts, and equivalents of all means or step plus function elements, if any, in the claims below are intended to include any structure, material, or act for performing the function in combination with other claimed elements as specifically claimed.

The description has been presented for purposes of illustration and description, but is not intended to be exhaustive. Many modifications and variations will be apparent to those of ordinary skill in the art without departing from the scope and spirit of the disclosure. The described aspect(s) was/were chosen and described in order to best explain the principles of the disclosure and the practical application, and to enable others of ordinary skill in the art to understand the disclosure for various aspects with various modifications as are suited to the particular use contemplated.

What is claimed is:

1. An odor removal system comprising:
a water-resistant housing comprising a cover and a base, the base having walls forming a compartment, the cover configured to cover the base when closed and expose the base when opened,
a wall of the base has a first opening, the first opening being an air inlet to the water-resistant housing, the first opening having installed a screen and/or a filter, the base further comprising another wall with a second opening, the second opening providing an outlet for air to leave the water-resistant housing;
at least one static bar configured to produce ions, the at least one static bar positioned in an airflow between the first opening and the second opening, wherein ion producing elements are integral to the at least one static bar; and
a centrifugal fan positioned adjacent to the second opening and attached to the another wall, the centrifugal fan having a fan outlet aligned with the second opening, the centrifugal fan configured to force the ions and the air into an external pipe connected with a target to neutralize odor within the target,
wherein power is selectively supplied to the at least one static bar and the centrifugal fan from an external power source.

2. The odor removal system of claim 1, wherein the at least one static bar is arranged in a first direction substantially orthogonal to the airflow within the water-resistant housing.

3. The odor removal system of claim 1, wherein the at least one static bar comprises two or more static bars, where the two or more static bars are substantially parallel to each other.

4. The odor removal system of claim 3, wherein each static bar is arranged in a first direction substantially orthogonal to the airflow within the water-resistant housing and is offset from each other in a second direction and a third direction, the second direction is substantially orthogonal to the first direction and the third direction.

5. The odor removal system of claim 1, further comprising at least one internet of things (IoT) device, each IoT device comprising a wireless communication interface and a sensor, where each IoT device is configured to wirelessly communicate a sensed value to a management system.

6. The odor removal system of claim 5, wherein the sensor is an ion sensor configured to sense a threshold amount of ions and wherein the wireless communication interface is configured to transmit an indication based on a sensing to the management system.

7. The odor removal system of claim 5, wherein the sensor comprises a clock configured to track a number of hours the at least one static bar is ON and wherein the wireless communication interface is configured to transmit the number of hours to the management system.

8. The odor removal system of claim 1, wherein the target comprises a tank selected from a group consisting of a wet well, a pump station, lift station, a sludge storage tank, a septic tank, and an equalization tank.

9. The odor removal system of claim 1, further comprising:
a sensor configured to detect a position of the cover; and
a controller configured to control the centrifugal fan to turn ON in response to the sensor detecting the position of the cover is closed, wherein the system further comprises a delay configured to delay the at least one static bar from turning ON until a time has elapsed from a time in which the centrifugal fan is turned ON.

10. The odor removal system of claim 9, further comprising an air quality sensor including a hydrogen sulfide sensor positioned adjacent to the at least one static bar, wherein when the hydrogen sulfide sensor detects hydrogen sulfide within the water-resistant housing above a threshold even after the time has elapsed, the at least one static bar is restricted from producing ions.

11. The odor removal system of claim 1, wherein the external pipe comprises a damper and wherein the system further comprises an actuator configured to control the damper to block airflow when the centrifugal fan is not turned ON.

12. The odor removal system of claim 1, further comprising a temperature sensor positioned adjacent the first opening or in the external pipe and configured to sense an ambient temperature of airflow and wherein the system further comprises a heater, and wherein when the ambient temperature is less than or equal to a threshold, the heater is controlled to heat the airflow until the ambient temperature is above the threshold.

13. The odor removal system of claim 5, wherein the sensor is an air quality sensor in air communication with the target and wherein a speed of the centrifugal fan is controlled based on a detection result from the air quality sensor.

14. The odor removal system of claim 1, wherein the cover is a door rotatably mounted to the base, and wherein the door comprises a plurality of indicating devices including a first indicating device indicating whether the odor removal system is receiving the external power and a second indicating device indicating whether the at least one static bar is ON.

15. The odor removal system of claim 1, further comprising a plurality of radio frequency identification devices (RFID), each RFID device having a sensor, wherein in response to an interrogation by a reader, each RFID device is configured to transmit a sensed value to the reader.

16. The odor removal system of claim 1, further comprising a stand connectable to the wall of the base having the first opening, the stand comprising a plurality of legs to support the water-resistant housing, where the legs do not overlap the first opening.

17. The odor removal system of claim 1, wherein each static bar is arranged in a first direction substantially orthogonal to an airflow within the water-resistant housing and is offset from each other in either a second direction or a third direction, the second direction is substantially orthogonal to the first direction and the third direction.

18. The odor removal system of claim 5, wherein the sensor is a differential pressure sensor arranged and configured to detect pressure drop across the filter, wherein in a case where the detected pressure drop is above a threshold, the wireless communication interface is configured to transmit in indication to clean or replace the filter to the management system.

19. The odor removal system of claim 1, wherein the at least one static bar is arranged at a non-parallel, non-orthogonal angle to the wall that has the first opening.

20. An odor removal system comprising:
a water-resistant housing comprising a cover and a base, the base having walls forming a compartment, the cover configured to cover the base when closed and expose the base when opened,
a wall of the base has a first opening, the first opening being an air inlet to the water-resistant housing, the first opening having installed a screen and/or a filter, the base further comprising another wall with a second opening, the second opening providing an outlet for air to leave the water-resistant housing;
at least one static bar configured to produce ions, the at least one static bar positioned in an airflow between the first opening and the second opening, wherein ion producing elements are integral to the at least one static bar; and
a fan positioned adjacent to the second opening and attached to the another wall, the fan having a fan outlet aligned with the second opening, the fan configured to force the ions and the air into an external pipe connected with a target to neutralize odor within the target,
wherein power is selectively supplied to the at least one static bar and the fan from an external power source.
